(19) United States
(12) Patent Application Publication
(10) Pub. No.: US 2011/0218448 A1
(43) Pub. Date: Sep. 8, 2011

(54) PERFUSION DETECTION DEVICES AND METHODS OF USING THE SAME

(76) Inventor: Rudolf F. Buntic, Burlingame, CA (US)

(21) Appl. No.: 12/717,056

(22) Filed: Mar. 3, 2010

(51) Int. Cl. A61B 5/026

(52) U.S. Cl. 600/504

(57) ABSTRACT

Hand-held pocket-sized self-contained perfusion detection devices are provided. Devices of the invention are configured to rapidly provide a perfusion detection result without the need for pre-calibration. Also provided are methods of using the perfusion detection devices, as well as kits that include the devices and/or components thereof for use in practicing such methods.

Following various types of surgery, it is important that proper blood flow is maintained in the body. Surgeries for replanted parts (e.g., fingers, hands, arms, facial parts, scalps, etc.) and microvascular transplants (also known as “free flaps” or “flaps”) often involve one or two small inflow arteries and one or two small outflow veins. Microsurgery, also referred to as microvascular surgery, is surgery that is performed on very small structures, such as blood vessels, using specialized equipment and a microscope. For instance, microsurgery may involve blood vessel repair and/or vein grafting. Monitoring of blood flow, both arterial inflow and venous outflow, is especially critical after microsurgery. Any compromise in circulation of the arterial inflow or venous outflow can lead to loss of the replanted part or flap.

Early detection of arterial or venous compromise can be critical to salvaging replants and flaps, and to prevent failure. As blood is circulated throughout the body, it perfuses into tissue to supply the cells with nutrients and oxygen needed for the tissue to survive. Early detection of perfusion disturbances results in rapid initiation of remedial therapy to reverse circulatory compromise. Remedial therapy may include removing mechanical obstruction to flow, such as tight dressings, pharmacologic therapy such as anticoagulants and medicinal leeching, and/or surgical intervention and establishment of reflow of vascular occlusions.

### SUMMARY

Hand-held pocket-sized self-contained perfusion detection devices are provided. Devices of the invention are configured to rapidly provide a perfusion detection result without the need for pre-calibration. Also provided are methods of using the perfusion detection devices, as well as kits that include the devices and/or components thereof for use in practicing such methods.

### BRIEF DESCRIPTION OF THE FIGURES

FIGS. 1A and 1B provide front and side views, respectively, of a perfusion detection device according to an embodiment of the invention.

FIGS. 1C and 1D provide front and side views, respectively, of a perfusion detection device according to another embodiment of the invention.

FIG. 2 provides a functional block diagram illustrating different circuitry functional components of the device illustrated in FIGS. 1A and 1B.

FIG. 3 illustrates a flowchart of a method of detecting perfusion, according to some embodiments.

FIG. 4 illustrates a portion of a circuit schematic including a photodiode and signal amplifier for a perfusion detection device, according to an embodiment of the invention.

FIG. 5 illustrates a portion of a circuit schematic including a lock-in amplifier, root mean square converter, and low pass filter for a perfusion detection device, according to some embodiments of the invention.

FIG. 6 illustrates a portion of a circuit schematic including a microcontroller for a perfusion detection device, according to some embodiments of the invention.

### DETAILED DESCRIPTION

Hand-held pocket-sized self-contained perfusion detection devices are provided. Devices of the invention are configured to rapidly provide a perfusion detection result without the need for pre-calibration. Also provided are methods of using the perfusion detection devices, as well as kits that include the devices and/or components thereof for use in practicing such methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the invention, aspects of embodiments of the subject perfusion detection devices and systems are described first in greater detail. Next, embodiments of methods of detecting perfusion of a target tissue in which the subject perfusion detection devices and systems may find use are reviewed in greater detail.

### PERFUSION DETECTION DEVICE

As summarized above, aspects of the invention include perfusion detection devices. Devices of the invention are configured to rapidly provide a perfusion detection result without the need for pre-calibration. A perfusion detection result is an output that may be employed by the user in the evaluation or assessment of perfusion in the target tissue location, e.g., to determine whether adequate perfusion is or is not present in the target tissue location. The nature of the perfusion detection result may vary, where the result may be qualitative (e.g., sufficient or insufficient or colors indicating the same, such as green, yellow or red) or quantitative, e.g., in the form of a numerical value that represents the amount or level of perfusion in the target tissue location. As such, the devices may be employed to obtain a perfusion detection result without first calibrating the device. Accordingly, the devices of the invention may be employed with out first checking and/or adjusting the device function with a standard sample.

Devices of interest include a light source configured to deliver an excitation wavelength of light to a fluorescent agent during use. Light sources of interest may vary, so long as they are capable of exciting the fluorescent agent that is being used in the perfusion detection methods being performed with the device. Of interest are light sources that need not be calibrated prior to use, such that they need not be checked with reference or standard prior to use. Light sources of interest therefore exhibit consistent brightness (e.g., such that do not fade to any appreciable extent), over the lifetime of the device, which may be at least 6 months or longer, such as 1 year or longer, including 5 years or longer. Any fading that does occur is minimal, being no more than 10%, such as no more than 5%. In some instances, the light source is bright, where the brightness may range in some instances from 200 to 10,000 millicandelas (mcd), such as 200 to 5,000 mcd. In addition, light sources of interest produce low amounts of heat and have low power consumption. Of interest are light sources that consume 1 watt or less, such as ½ watt or less, and including ⅕ watt or less. In some embodiments, light sources of interest have an energy conversion of at most 1/10 watt (e.g., a blue-emitting LED with energy conversion of less than 1/10 watt).

Of interest as light sources in devices of the invention are light emitting diodes (LEDs). LEDs of interest may be configured to receive a periodic signal having a certain frequency, and thus be modulated based on the periodic signal. The periodic signal may be of various waveforms—e.g., sine, pulse (e.g., square), triangle, sawtooth, etc. In some embodiments, the periodic signal is a pulse-wave and the LED is pulsed on and off at a frequency of the pulse-wave. Further, the frequency of the periodic signal may be set to a variety of frequencies, and in some instances, is set to a frequency in the range of less than 1 Hz to several MHz, such as 1 Hz to 1 MHz, including 5 Hz to 50 Hz. In some embodiment, the frequency is set to a range between 15 and 25 Hz.

Various types of LEDs may be implemented depending on specific application and design considerations (e.g., color of LED, intensity, etc). For example, various LEDs may be used to emit different wavelength light corresponding to different colors, e.g., 450-500 nm wavelength corresponding generally to blue LEDs. LEDs of interest include, but are not limited to, those that emit light in wavelengths ranging from visible light to infrared, e.g., from 450 nm to 950 nm, among others. Of interest in some instances are LEDs that emit light in a narrow wavelength band range, e.g., a band range that is 100 nm or less, such as 75 nm or less, including 50 nm or less. Furthermore, turn-on voltages for the LED may vary from color to color. For example, blue LEDs may require around 2.4 to 3.7 volts (although such range of voltage drop is exemplary and some blue LEDs may require voltage drops outside of that range). In some embodiments, the perfusion detection devices include a blue LED to illuminate the target tissue site. Where desired, the device may be configured so that the LED is interchangeable, such that the LED may be removed and replaced with another LED without compromising the structural or functional configuration of other components of the device.

In some instances, the LED may be interchangeable so that a new LED may be used in place of the existing LED. In this way, not only old and/or damaged LEDs may be replaced, but also, different types of LEDs may be used instead. For example, an LED of a different wavelength (e.g., different color) and/or intensity limits may be used in place of the current LED. In such case, the optional emission light filter, described in further detail, may also be interchanged to account for the new color LED.

The perfusion detection devices also include a processor. It should be understood that the term processor is used herein to generally refer to any processing device such as a microprocessor, microcontroller, and/or digital signal processor. The processor may be configured to execute various functions, such as various functions of functional blocks, and/or techniques discussed herein. Furthermore, memory, such as RAM, ROM, flash, etc., may be coupled to the processor (and/or located within the processor) and store software program code and data associated with the present invention. The processor, in turn, comprises processing elements and/or logic circuitry to execute the software code.

The perfusion detection devices may further include a wave generator to generate the periodic signal (e.g., a pulse-wave) that is provided to the LED. In some instances, a processor executes the function of the wave generator. In some embodiments, the wave generator may comprise a discrete oscillator (e.g., external to the processor).

The wave generator may also provide the above-mentioned periodic signal to a lock-in amplifier as a reference signal. For example, a lock-in amplifier may be configured to receive a pulse-wave reference signal from the wave generator (e.g., the pulse-wave signal provided by a processor to the LED).

The wave generator may also be configured to allow the frequency of the periodic signal to be programmably adjusted. For example, the processor may be programmable such that the frequency of the pulse-wave generated by the wave generator may be adjusted according to software. For example, the frequency of the periodic signal may be programmably adjusted to a variety of frequencies, and in some instances, may be adjusted to a frequency between less than 1 Hz to several MHz, such as 1 Hz to 1 MHz, including 5 Hz to 50 Hz. In some embodiment, the user settings allow the user to adjust the frequency of the periodic signal to a frequency between 15 and 25 Hz.

The perfusion detection devices may further include an optional emission filter positioned between the LED and an emission fiber optic to pass essentially the color of light emitted by the LED. For example, a blue emission filter may be used to pass blue light from a blue LED. In such case, light with wavelengths outside of the band of the filter are filtered out. While the band of the filter may vary, filter bands of interest include, but are not limit to: 425-475 nm, 830-840 nm, etc.

An emission fiber optic may also be included in the perfusion detection devices and configured to receive light emitted from the LED (and filtered by the emission light filter if implemented) and to direct it to the target tissue site. For example, the emission fiber optic may extend from the LED (or emission filter if implemented) to an aperture in the housing of the perfusion detection device. In such case, the light emitted from the LED is directed through the emission fiber optic, out the aperture, and to the target tissue site or location. In some embodiments, the emission fiber optic comprises a bundle of fiber optics that guide the light from the LED to the target tissue site. In some instances, the bundle of fiber optics may be positioned, for example, around a receiving fiber optic. It should be understood that in some embodiments, other types of optical waveguides (e.g., rectangular waveguides, etc.) may be implemented in place of the emission fiber optic and/or receiving fiber optic.

The perfusion detection devices may also include a receiving fiber optic, e.g., which is configured to receive the fluorescent light from the target tissue site and direct it to the detector. For example, the receiving fiber optic may extend from an aperture in the housing of the perfusion detection device to or near a detector, e.g., a photodiode. In this way, fluorescent light is directed to the detector. In some instances, the receiving fiber optic a single optical fiber. In some instances, the receiving fiber optic is a bundle of multiple optical fibers.

The perfusion detection devices may also include a receiving light filter positioned between the receiving fiber optic and the detector to pass essentially the color of the fluorescent light of interest and prevent passage of other light. Light with wavelengths outside of the band of the receiving light filter will be rejected—e.g., ambient light. The wavelength of the fluorescent light emitted from the fluorescent agent will vary depending on the specific fluorescent agent and color of LED implemented. The receiving light filter that is implemented in the device is based on the particular wavelength of the fluorescent light emitted from the fluorescent agent. For example, when using fluorescein and a blue LED, the fluorescent light produced may have a wavelength in or near the yellow-green region. Accordingly, for this example, a yellow-green receiving filter is present in the device to pass the yellow-green fluorescent light and to filter out other colored light. In certain embodiments, some noise may also be passed by the receiving light filter (e.g., ambient light in the room having wavelengths in the band of the filter). While the band of the receiving light filter may vary, bands of interest include, but are not limited to: 515 to 535 nm; 780 to 810 nm, etc.

Also present in devices of the invention are light detectors. The detector is a transducer used to convert light into electrical energy so as to produce an output signal representative of the modulated fluorescent light. It should be understood that the term “representative of” is used herein to mean that the frequencies of the output signal and light are substantially the same and the amplitudes of the output signal and light are proportional.

In some embodiments, a photodiode is used to convert the received light into current, such that the detector is a photodiode. The current produced at the output of the photodiode is representative of the fluorescent light received by the photodiode. Thus the current is also modulated (e.g., pulsed) at the same frequency as the fluorescent light and the LED. In some instances, noise may also be present in the current signal output by the photodiode. Nose that may be present includes any noise entering the photodiode as well as any electrical noise generated by the photodiode. For example, noise may include fluorescent lights in a room (e.g., pulsing at 120 times per second), incandescent room lights (essentially steady state light), sunlight (essentially steady state light), etc.

The perfusion detection devices may also include a signal amplifier coupled to the detector. The signal amplifier may be used to amplify the signal output from the detector. In some instances, the signal amplifier includes a current to voltage converter coupled to a voltage amplifier. The current to voltage converter receives the current signal output by the photodiode and converts it to an output voltage signal representative of the current signal. The voltage amplifier receives the output voltage signal from the current to voltage converter and provides an amplified voltage signal representative of the current signal output by the photodiode. Accordingly, the amplified voltage output is representative of the current output by the photodiode, as well as the fluorescent light and LED. In certain embodiments, some noise may also be present in the amplified signal output by the signal amplifier. This includes any noise already in the signal output by the detector, as well as, any electrical noise generated by the signal amplification. Where desired, good low-noise amplifiers are employed.

The perfusion detection device may also include a lock-in amplifier that is configured to receive the signal output from the detector (and signal amplifier when implemented). For example, in some instances, the lock-in amplifier may be coupled to the signal amplifier and receive the amplified voltage signal output by the signal amplifier and detector. The lock-in amplifier uses a technique known as phase sensitive detection to single out a component of the signal at a specific reference frequency and phase. In this way, noise signals, at frequencies other than the reference frequency, may be rejected and do not affect the measurement.

The lock-in amplifier may also be configured to receive a reference signal at the frequency of interest (e.g., at the frequency that the LED is modulated). In some embodiments, the periodic signal (e.g., pulse-wave) that is generated by the wave generator and provided to the LED is also provided to the lock-in amplifier as a reference signal.

In some aspects, the lock-in amplifier functions to amplify the input signal received from the detector (or signal amplifier if implemented) and multiply it by a reference signal at the frequency of interest (e.g., the frequency at which the LED is modulated), resulting in a demodulated signal including a DC component that is proportional to the amplitude of the input signal received from the detector.

In certain embodiments, the lock-in amplifier may obtain the DC output component that is proportional to the amplitude of the input signal as follows. When two AC signals are multiplied together, the result is two signals at the sum and difference frequencies. It should be noted that the input signal received by the detector may include noise at other frequencies. For example, noise may include fluorescent lights in a room (e.g., pulsing at 120 times per second). Therefore, any input signals that are not at the same frequency as the reference signal will result in two AC signals. Any input signals at the same frequency as the reference signal will result in an AC signal (sum) and a DC signal (difference). (Thus, a lock-in amplifier may be thereafter coupled to a low pass filter to pass the DC signal and to substantially filter out the AC signals. It should be understood that the DC signal is not a pure DC signal as noise at frequencies very close to the reference frequency will result in very low frequency AC outputs. Their attenuation depends upon the low pass filter bandwidth and rolloff. Thus, narrowband filters may be present to remove more of the noise sources very close to the reference frequency.).

The resulting DC output signal is proportional to the product of the amplitude of the input signal received from the detector and the cosine of the phase difference between the input signal and the reference signal. Therefore, by adjusting the phase difference between the two signals to be zero, the DC output signal will be proportional to the amplitude of the input signal received from the detector.

It should be understood that various lock-in amplifiers may be implemented. For example, digital or analog lock-in amplifiers may be implemented in various embodiments. It should be understood that in embodiments where a digital lock-in amplifier is used, the output signal from the detector has to be converted to a digital signal (e.g., by an analog to digital converter). Furthermore, in some embodiments, a second phase sensitive detector may be implemented to eliminate the phase dependency described above. For example, the second phase sensitive detector may multiply the input signal with the reference signal shifted by 90 degrees, thus resulting in an output signal proportional to the product of the amplitude of the input signal received from the detector and the sine of the phase difference between the input signal and the reference signal. In such case, both output signals from the phase sensitive detectors may together be treated as a vector and the resulting magnitude calculated (e.g., by a processor) thus removing the phase dependency. The resulting magnitude is the amplitude of the input signal received from the detector.

The perfusion detection devices may also include a root mean square converter configured to receive the demodulated signal from the lock-in amplifier. Because lock-in amplifiers generally detect only root mean square signals at the frequency of interest, perfusion detection devices may also include a root mean square converter configured to receive the demodulated signal from the lock-in amplifier and multiply it by the necessary factor (e.g., square root of two) to produce a output signal with a peak value as opposed to the root mean square value. In some instances, the root mean square converter provides the advantage of preserving more of the signal. (Rudy-please expand on how this is achieved).

In some instances, the perfusion detection devices may also include a low pass filter coupled to the root mean square converter to receive the demodulated signal output by the root mean square converter. The low pass filter is configured to pass the

DC component of the demodulated signal that is proportional to the signal from the detector and to filter out the AC component signals at other frequencies. In some instances, the DC signal is not a pure DC signal as noise at frequencies very close to the reference frequency will result in very low frequency AC outputs. Their attenuation depends upon the low pass filter bandwidth and rolloff. Thus, narrowband filters may be used to remove more of the noise sources very close to the reference frequency.

The perfusion detection devices may further include an analog to digital converter (A/D converter) configured to receive a signal from the low pass filter and output a digital signal to a functional block that converts the digital signal to the perfusion detection result. The perfusion detection result may then be sent to a display for display to the user. In some instances, the A/D converter may be a discrete solid state component coupled to a processor, for example. In some instances, the processor may perform the function of the ND converter and convert the received analog signal to a digital signal.

In some instances, the perfusion detection devices may include additional functional blocks for performing various other functions as desired, such as interpreting the strength of the fluorescent light, changing the interpretation of light emitted from the LED (e.g., LED brightness, modulation rate, etc.), monitoring battery level, indicating low battery level, power savings, etc. In some embodiments, the functional block may be configured to simultaneously generate the pulse-wave and interpret the strength of the fluorescent light. Thus, the devices may include software controlled analysis, for example, of fluorescence and software adjustment of fluorescence display.

Furthermore, the perfusion detection device may include a functional block for performing adjustments to program settings of the device according to various user inputs. For example, the device may be programmed according to user inputs to adjust settings such as scale, screen brightness, etc. It should be understood that the functions of the functional blocks described herein may, in some instances, be executed by a processor.

In some aspects of the invention, the processor is configured to provide a perfusion detection result in 1 minute or less following illumination of a target tissue site with light from the LED without pre-calibration. For example, the perfusion detection result is obtained, in some instances, within 30 seconds or less, such as 10 seconds or less. In some instances, the perfusion detection result is obtained substantially immediately, such as 5 seconds or less, following illumination of the target tissue site with the LED.

Perfusion detection devices include a power source. The power source supplies power to the perfusion detection device. The power source may comprise one or more DC batteries—e.g., 4.5 volt, 9-volt, D, C, AA, AAA batteries, etc. In some embodiments, the perfusion detection device may be powered by a remote battery pack or AC source via a cable. Power may be supplied by the power source through a switch, for example, which allows the device to be powered on and off by the user. Furthermore, it should be understood that the perfusion detection devices may further include one or more voltage regulators coupled to the power source to provide the voltage supply levels needed by various operational components.

As summarized above, in some aspects of the invention, embodiments of the perfusion detection devices include a self-contained pocket-sized housing. The housing may include, for example, operational components such as a power source, light emitting diode (LED), detector, processor, and/or other operational components as described herein. The term “operational components” is used herein to refer to generally to any electrical, mechanical, and optical components used for operation of the device. The housing may be made of a variety of materials such as plastics, other synthetic materials, metals, metal alloys, etc.

The perfusion detection devices are dimensioned such that the devices are hand-held and may be introduced to the target tissue location on a subject while being held. As the devices, the may be small, lightweight, and portable in order to be operated easily and conveniently by the user. For hand-held devices, the devices are dimensioned to be held comfortably and easily by the user, where dimensions of specific embodiments may vary. For example, the length of the device may vary, and in some instances may be 10 inches or less, such as 8 inches or less, and including 7 inches or less, where in some instances the length of the device ranges from 1 to 10 inches, such as 2 to 8 inches and including 2 to 7 inches. Similarly the width of the device may vary, and in some instances may be 8 inches or less, such as 5 inches or less, and including 2 inches or less, where in some instances the width of the device ranges from 0.5 to 5.0 inches, such as 1 to 3 inches and including 1.5 to 2.5 inches. Furthermore, the thickness of the device may vary, and in some instances may be 5 inches or less, such as 3 inches or less, and including 2 inches or less, where in some instances the thickness of the device ranges from 0.25 to 2.5 inches, such as 1 to 1.5 inches. As the device is hand-held, the weight of the devices is such that the devices may be operably hand-held during use. While the weight of the devices may vary, in some instances the weight of the devices is 3 lbs. or less, such as 2 lbs or less, and including 1 lb. or less, ranging in some instances from 0.25 to 3 lbs, such as 0.50 to 2 lbs. The perfusion detection devices may have a variety of shapes, such as rectangular, cylindrical, irregular shapes, etc.

The hand-held devices are also generally designed to be portable and in certain embodiments wireless. In some embodiments, the hand-held device is battery operated and un-tethered to promote complete portability and mobility. Various DC battery sizes may be implemented—e.g., one or more 4.5 volt, 9-volt, D, C, AA, AAA batteries, etc., as described above, where the power source may be rechargeable, as desired. In some embodiments, however, the hand-held device may be tethered to a remote battery pack or AC source.

In some instances, the perfusion detection devices include a housing that has a head section and body section. The head section pertains to the section of the device where the device interfaces with the target tissue site. As such, the head section may include an aperture and operational components relating to the delivery and reception of light waves. For example, the head section may include the receiving fiber optic, receiving light filter, emitting fiber optic, emitting fiber optics, and detector, in some instances. In some instances, the head section includes an ambient light shield to block some amounts of ambient light from reaching the detector. In some instances, the head section includes a speculum—e.g., a cylindrical or cone-shaped section of the housing with the aperture at the end—that functions as an ambient light shield.

The body section pertains to the section of the device where the user holds the device. In some instances, the body section of the device is larger than the head section of the device. In these embodiments, the body section may have a volume that exceeds that of the head section by 2 times or more, such as by 5 times or more, and including by 10 times or more. The body section may further include a circuit board and operational components such as an LED, display, amplifier, root mean square converter, power source, filters, control switches, control units, etc. It should be understood that some operational components may be placed either in the head section or the body section. For example, in some instances, the LED and/or display may be positioned in the head section of the device instead of the body section. In some embodiments, the body section includes at least a power source, LED, and processor, while the head section includes at least a detector.

In some instances, the head section is movable relative to the body section. The device may be configured so that the head moves in one or more directions relative to the body section. For example, the head section may rotate along one or more axes (i.e., x, y, or z axis) relative to the body section. In some instances, the head section may move freely in all directions, such as provided by a ball and socket type mechanism for example. Further, in some instances, the head section may translate outward or inward—i.e., extending towards or away from the device. A mechanical connector may be coupled to the head section and body section to enable the movement of the head section relative to the body section. In some instances, the mechanical connector includes a channel such that one or more components (e.g., fiber optics and/or electrical wires) may be extended from one section to the other.

In some instances, the device is configured to define a specific area for the device to interface with the target tissue site so as to ensure that fluorescent light received in the aperture more closely corresponds to the target tissue that is excited. Accordingly, devices of the invention may be configured to emit LED light and receive fluorescent light (from the fluorescent agent) through a common aperture. In some instances, the device may include an ambient light shield configured to shield the detector from ambient light from outside the detector. In some embodiments, the ambient light shield is a speculum. For example, the housing may include a speculum—e.g., cylindrical or cone shaped section of the housing (also referred to herein as the “nose” of the housing)—and the aperture located at the end of the speculum. The may assist in blocking out some ambient light from entering the device and reaching the detector.

Furthermore, in some instances, the device may include a disposable speculum piece that is removably coupled to the housing where the LED light is emitted and the fluorescent light is received. For example, the disposable speculum piece may be configured to be removably coupled to the speculum (e.g., cylindrical or cone-shaped section of the housing). In such cases, the aperture in the disposable speculum piece is aligned with the aperture in the cylindrical or cone-shaped section so as to not block the LED light emitted or the fluorescent light received. The disposable speculum piece allows for the sanitary use of the device. For example, the disposable speculum piece prevents the perfusion detection device from contacting the target tissue site and becoming contaminated. The disposable speculum may, for example, be coupled and discarded by the user for each reading. The disposable speculum piece may be made of a various non-conductive materials such as plastic, latex, rubber, other synthetic materials, etc.

In some instances, the perfusion detection devices may include electromagnetic interference (EMI) shielding to protect one or more components from electromagnetic disturbances. In some embodiments, the signal amplifier and photodiode are present in an EMI shield. The EMI shielding may be, for example, various conductive materials such as metals, metal alloys, etc. In some instances, the EMI shielding may be provided by a non-conductive material (e.g., plastic) that is coated with conductive materials (e.g., conductive ink).

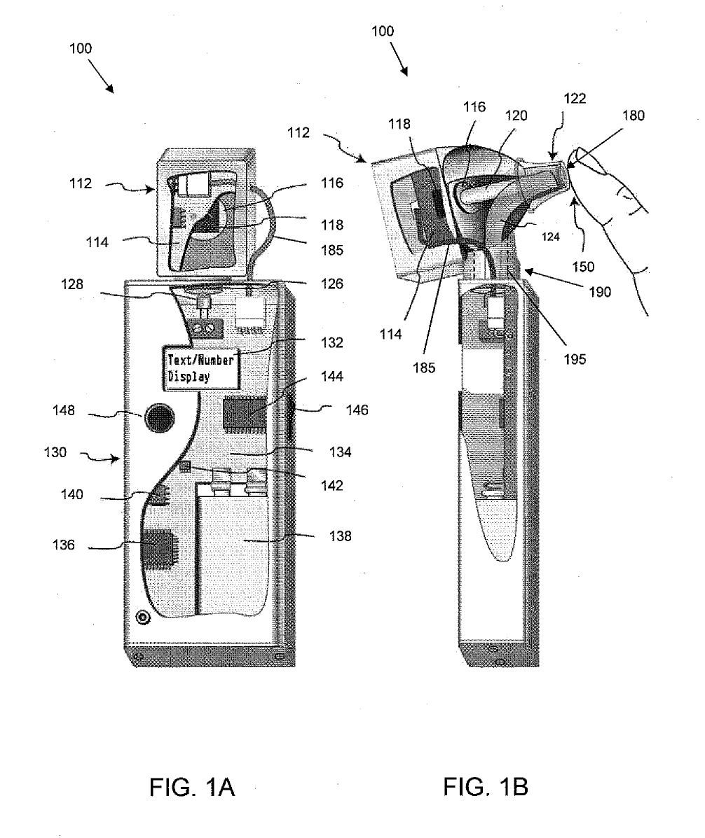

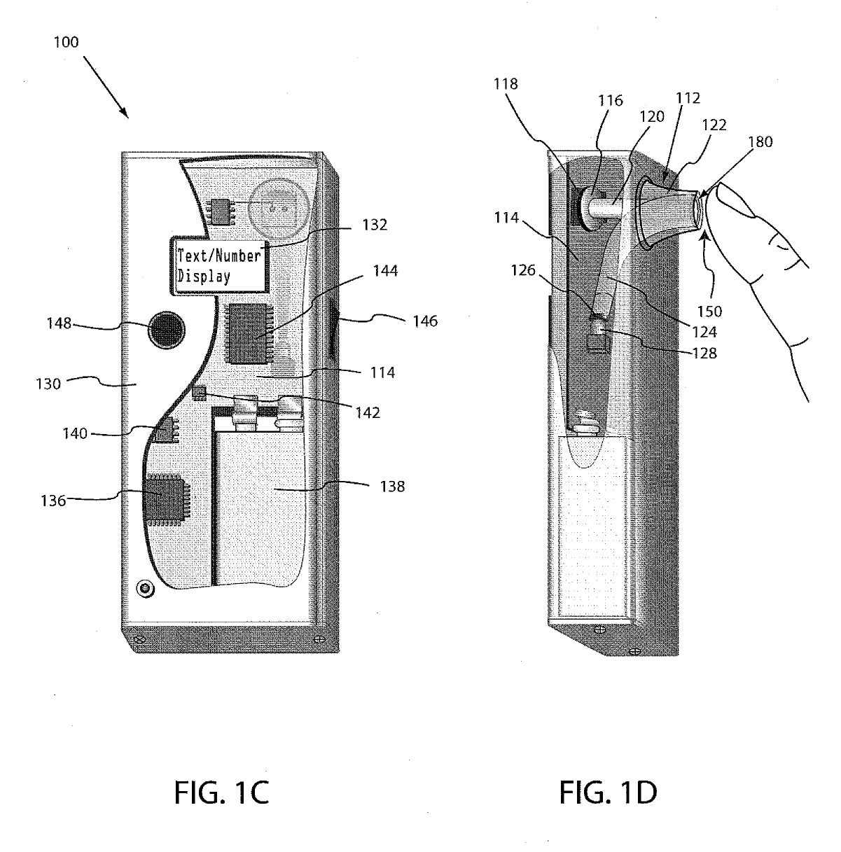

Turning to the figures, FIGS. 1A and 1B illustrate a top and side view, respectively, of a hand-held perfusion detection device 100, according to some embodiments. The hand-held perfusion detection device 100 is shown to include a head section 112 and a body section 130. The head section 112 as illustrated in FIGS. 1A and 1B is configured to interface with a target tissue site 150 and is shown to include the emission fiber optic 124, receiving fiber optic 120, receiving light filter 116, and circuit board 114 including detector 118 (e.g., a photodiode). The emission fiber optic 124 is positioned to receive light from an LED 128 (shown in the body section 130 of FIGS. 1A-1B) and extend to an aperture 180 in the speculum or nose of the head section 112 (shown covered by a disposable speculum 122). In some embodiments, the emission fiber optic 124 is a fiber optic bundle that surrounds at least a portion (such as the distal portion) of the receiving fiber optic 120. The disposable speculum 122 prevents the head section 112 from contacting the target tissue site 150 and becoming contaminated. The disposable speculum 122 may, for example, be coupled by the user for each reading and then discarded.

The receiving fiber optic 120 is positioned in the head section 112 and extends from the aperture 180 in the speculum of the head section 112 to the receiving light filter 116. The receiving light filter 116 is positioned between the receiving fiber optic 120 and the detector 118 (e.g., photodiode). The detector 118 is coupled to circuit board 114 and a wire 185 electrically couples the detector 118 to circuit board 134 within the body section 130 of the housing.

The head section 112 includes a mechanical connector 190 that couples the head section 112 to the body section 130. The mechanical connector 190 includes a channel 195 (shown in dotted lines) in which the emission fiber optic 124 and wire 185 may pass. The emission fiber optic 124 is shown extending up to the body section 130 and terminating adjacent to the LED 128 and optional emission light filter 126. Wire 185 passes through the channel 195 and into the body section 130 to the circuit board 134. In some instances, the mechanical connector 190 is configured to enable the head section 112 to rotate and/or translate, such as described earlier.

Body section 130 is shown to include circuit board 134 and various operational components. The LED 128 is positioned on the circuit board 134 near the emission fiber optic 124 cable so that light emitted from the LED 128 travels though the emission fiber optic 124. An optional emission light filter 126 is shown positioned between the LED 128 and the emission fiber optic 124. The optional emission light filter 126 may be, for example, a filter corresponding to the wavelength of the light emitted from the LED 128 so as to pass the light from the LED 128 but filter out other wavelengths of light. In some embodiments, the LED 138 and/or optional emission light filter 126 are interchangeable so that a new LED and/or optional emission light filter may replace the existing one.

The circuit board 134 within the body section 130 of the housing is shown to also include a lock-in amplifier 144, root mean square converter 142, and low pass filter 140.

The circuit board 134 within the body section 130 is shown to further include processor 136 (e.g., a microcontroller) and power source (e.g., a DC battery).

Body section 130 also includes a display 132 coupled to the circuit board 134 and positioned on the body section 130 of the housing so that the display 132 may be viewed externally by a user. Control switches such as a user input element 148 (e.g., a toggle switch) and power switch 146 are coupled to the circuit board 134 and positioned on the housing of the body section 130 for the user to operate. It should be understood that the control switches may be any type of control element such as a key, button, switch, dial, knob, etc.

Turning now to FIGS. 1C and 1D, illustrated in these figures is a top and side view, respectively, of a hand-held perfusion detection device 100, according to another embodiment of the invention. The hand-held perfusion detection device 100 is shown to include a head section 112 and a body section 130. The head section 112 as illustrated in FIGS. 1C and 1D is configured to interface with a target tissue site 150 and is shown to include the emission fiber optic 124, receiving fiber optic 120, receiving light filter 116, and circuit board 156 including detector 118 (e.g., a photodiode). The emission fiber optic 124 is positioned to receive light from an LED 128 (shown in the body section 130 of FIGS. 1C-1D) and extend to an aperture 180 in the speculum or nose of the head section 112 (shown covered by a disposable speculum 122). In some embodiments, the emission fiber optic 124 is a fiber optic bundle that surrounds at least a portion (such as the distal portion) of the receiving fiber optic 120. The disposable speculum 122 prevents the head section 112 from contacting the target tissue site 150 and becoming contaminated. The disposable speculum 122 may, for example, be coupled by the user for each reading and then discarded.

The receiving fiber optic 120 is positioned in the head section 112 and extends from the aperture 180 in the speculum of the head section 112 to the receiving light filter 116. The receiving light filter 116 is positioned between the receiving fiber optic 120 and the detector 118 (e.g., photodiode). The detector 118 is coupled to circuit board 114 within the body section 130 of the housing.

The emission fiber optic 124 is shown extending up to the body section 130 and terminating adjacent to the LED 128 and optional emission light filter 126. The head section can be positioned anywhere on the body, bottom, top, side or ends. It can placed in a separate casing from the body and connected to the body by wire and/or fiber optic cable. Body section 130 is shown to include circuit board 114 and various operational components. The LED 128 is positioned on the circuit board 114 (pictured here on the bottom surface, but it can be positioned on the top surface) near the emission fiber optic 124 cable so that light emitted from the LED 128 travels though the emission fiber optic 124. An optional emission light filter 126 is shown positioned between the LED 128 and the emission fiber optic 124. The optional emission light filter 126 may be, for example, a filter corresponding to the wavelength of the light emitted from the LED 128 so as to pass the light from the LED 128 but filter out other wavelengths of light. In some embodiments, the LED 128 and/or optional emission light filter 126 are interchangeable so that a new LED and/or optional emission light filter may replace the existing one.

The circuit board 114 within the body section 130 of the housing is shown to also include a lock-in amplifier 144, root mean square converter 142, and low pass filter 140. The circuit board 114 within the body section 130 is shown to further include processor 136 (e.g., a microcontroller) and power source (e.g., a DC battery).

Body section 130 also includes a display 132 coupled to the circuit board 134 and positioned on the body section 130 of the housing so that the display 132 may be viewed externally by a user. Control switches such as a user input element 148 (e.g., a toggle switch) and power switch 146 are coupled to the circuit board 114 and positioned on the housing of the body section 130 for the user to operate. It should be understood that the control switches may be any type of control element such as a key, button, switch, dial, knob, etc.

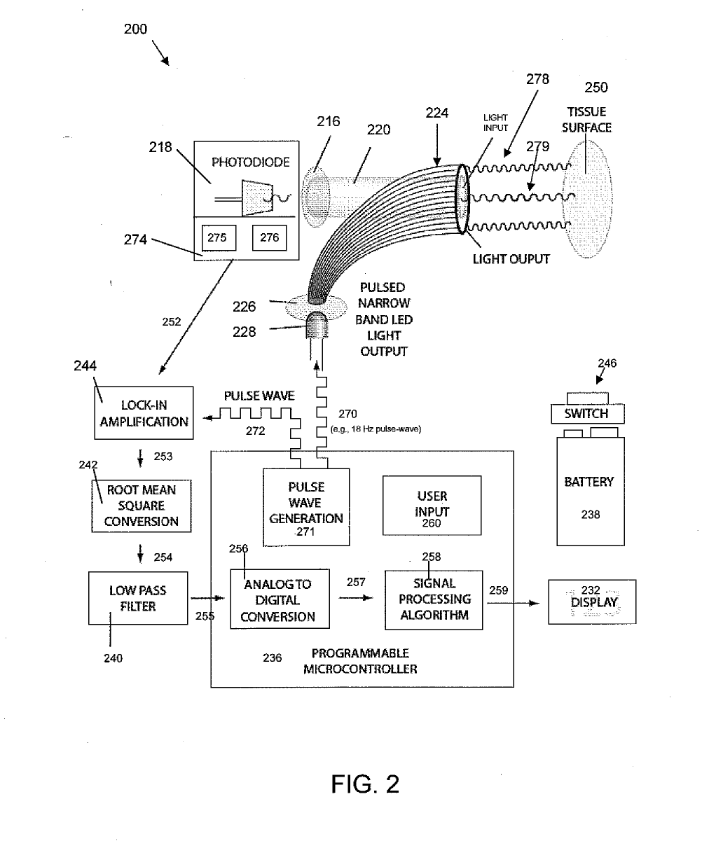

FIG. 2 provides a functional block diagram illustrating different circuitry functional components of the device illustrated in FIGS. 1A and 1B, according to some embodiments. As shown, perfusion detection device 200 includes a power source 238 for supplying power to the perfusion detection device 200. The power source 238 may comprise one or more DC batteries—e.g., 4.5 volt, 9-volt, D, C, AA, AAA batteries, etc. Switch 246 is coupled to the power source 238 and enables the device 200 to be powered on and off. Furthermore, it should be understood that the perfusion detection device 200 may further include one or more voltage regulators coupled (not shown) to the power source 238 to provide the voltage supply levels needed by various operational components.

As shown, the perfusion detection device 200 further includes an LED 228—e.g., a blue LED. The LED 228 is used to emit light that illuminates the target tissue site 250 and excites any fluorescent agent (e.g., fluorescein) diffused in the target tissue 250. The LED 228 is configured to receive a pulse-wave 270 generated by the microcontroller 236 (represented in FIG. 2 by wave generator 271) and thus is modulated based on the pulse-wave 270. LED 228 is pulsed on and off at a frequency of the pulse-wave. The frequency of the pulse-wave 270 may be set to a variety of frequencies, and in some instances, is set to a frequency in the range of less than 1 Hz to several MHz, such as 5 Hz to 100 Hz, including 10 Hz to 50 Hz. In the example shown in FIG. 2, the frequency is set 18 Hz. In some embodiments, the frequency may be programmably adjusted by software.

As shown in FIG. 2, the pulse-wave 270 generated by wave generator 271 is also provided to a lock-in amplifier 244 as pulse-wave reference signal 272. In some embodiments, the wave generator 271 is configured to allow the frequency of the pulse-wave 270 (and the pulse-wave reference signal 272) to be programmably adjusted—e.g., by software settings and/or user settings.

The perfusion detection device 200 is shown to further include an optional emission light filter 226 positioned between the LED 228 and an emission fiber optic 224 to pass essentially the color of light emitted by the LED 228. In some embodiments, the emission light filter 226 is a blue filter that passes blue light from a blue LED 228. In such case, light at other wavelengths than the emission light filter 226 are filtered out.

An emission fiber optic 224 is also shown included in the perfusion detection device 200. Emission fiber optic 224 is configured to receive light emitted from the LED 228 (and filtered by the emission light filter 226) and to direct it to the target tissue site 250. In the example embodiment shown in FIG. 2, the emission fiber optic 224 comprises a bundle of fiber optics that guide the light from the LED 228 to the target tissue site 250. Further, the bundle of fiber optics is positioned around a receiving fiber optic 220 at the distal end. It should be understood that in some embodiments, other types of optical waveguides (e.g., rectangular wave-guides, etc.) may be implemented in place of the emission fiber optic 224 and/or receiving fiber optic 220. The perfusion detection device 200 may also include a disposable speculum (not shown in FIG. 2) that is removably coupled to the perfusion detection device 200 so as to prevent the target tissue site 250 from contacting the perfusion detection device 200.

The light 278 from the LED 228 illuminates the target tissue site 250 and excites the fluorescent agent (e.g., fluorescein) that is diffused in the target tissue. As a result, the fluorescent agent fluoresces to emit light 279 towards the perfusion detection device 200. Because the light 278 from the LED 228 is modulated (e.g., pulsed) based on the pulse-wave 270, the fluorescent light is also modulated (e.g., pulsed) based on the pulse-wave 270.

Perfusion detection device 200 is shown to also include a detector 218 (shown in FIG. 2 as photodiode 218) and receiving fiber optic 220, which receives the fluorescent light from the target tissue site 250 and directs it to photodiode 218. In the embodiment shown, the receiving fiber optic 220 is a single fiber optic. In some embodiments, the receiving fiber optic 220 may be a bundle of fiber optics.

The perfusion detection device 200 is also shown to include a receiving light filter 216 positioned between the receiving fiber optic 220 and the detector to pass essentially the desired wavelength of the light 229 (e.g., yellow-green light). Light with wavelengths outside of the band of the receiving light filter 216 is rejected in the device shown in FIG. 2.

The photodiode 218 converts the received light from the receiving fiber optic 220 into a current signal. The current signal produced at the output of the photodiode 218 is representative of the emitted light 279 received by the photodiode. Thus the current is also modulated (e.g., pulsed) at the same frequency as the emitted light 279 and the LED 228. In certain embodiments, some noise may also be present in the current signal output by the photodiode 218. This includes any noise entering the photodiode 218, as well as, any electrical noise generated by the photodiode 218. For example, noise may include fluorescent lights in a room (e.g., pulsing at 120 times per second), incandescent room lights (essentially steady state light), sunlight (essentially steady state light), etc.

The perfusion detection device 200 is also shown to include a signal amplifier 274 coupled to the photodiode 218. The signal amplifier 274 amplifies the signal output from the photodiode 218. The signal amplifier 274 may include, for example, a current to voltage converter 275 coupled to a voltage amplifier 276. The current to voltage converter 275 receives the current signal output by the photodiode 218 and converts it to an output voltage signal representative of the current signal. The voltage amplifier 276 receives the output voltage signal from the current to voltage converter 275 and provides an amplified voltage signal 252 representative of the current signal output by the photodiode 218. Accordingly, the amplified voltage output 252 is also modulated (e.g.,. pulsing) at the same frequency as the current output by the photodiode 218, as well as the emitted light 279 and LED 228. In certain instances, some noise may also be present in the amplified signal output by the signal amplifier 274. This includes any noise already in the signal output by the detector 218, as well as, any electrical noise generated by the signal amplifier 274. In such instances, a low-noise amplifier sufficient to remove at least some of the noise may be employed, as desirable.

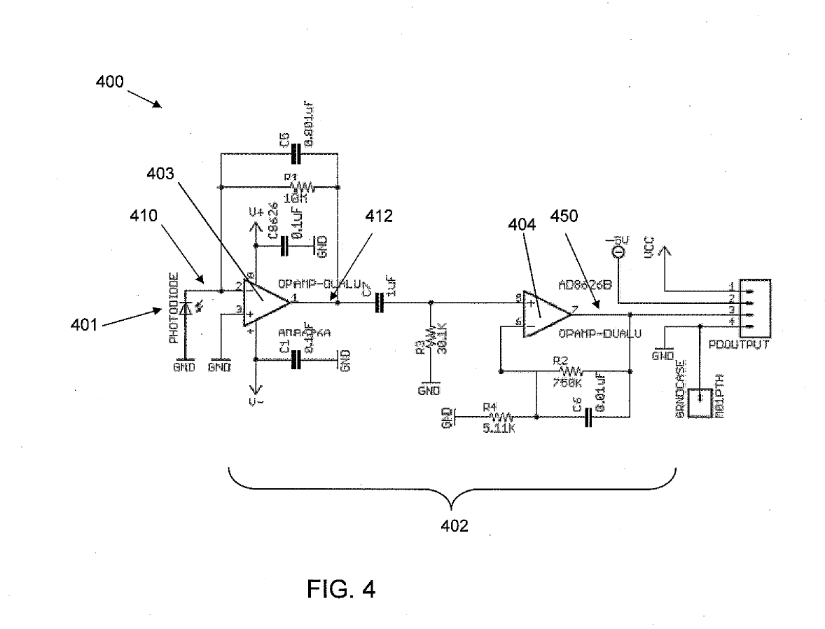

FIG. 4 illustrates a portion of a circuit schematic including a photodiode and signal amplifier for a perfusion detection device, according to some embodiments. Circuit part 400 is shown to include photodiode 401 and signal amplifier 402 which is represented by the remainder of the circuitry shown. Signal amplifier 402 includes operational amplifiers 403,404. Additional circuitry also shown in FIG. 4 includes various circuitry components provided for proper operation of the photodiode 401 and operational amplifiers 403,404—e.g., biasing circuitry, coupling circuitry, etc. Operation amplifier 403 is coupled to photodiode 401 at its input and is configured to generate a voltage output (as represented at reference numeral 412) representative of the current signal (as represented at reference numeral 410) generated by the photodiode when LED light is received. Operational amplifier 403 is coupled to operational amplifier 404 which is configured as a voltage amplifier. Operational amplifier 404 amplifies the voltage signal (as represented at reference numeral 412) generated by operational amplifier 403 and provides it as an output signal (as represented at reference numeral 450).

Turning back to FIG. 2, the perfusion detection device 200 further includes a lock-in amplifier 244 that is configured to receive the signal 252 output from the detector 218 and signal amplifier 274. Any convenient lock-in amplifier may be present. For example, an AD630 balanced modulator/demodulator by Analog Devices (Norwood, Mass.) may be implemented as lock-in amplifier 244. The lock-in amplifier 244 is also shown to receive pulse-wave reference signal 272 from the wave generator 271. The pulse-wave reference 272 signal is the same signal as the pulse-wave 270 that is generated by the wave generator and provided to the LED 228. The lock-in amplifier 244 amplifies the input signal 252 and multiplies it by the pulse-wave reference signal 272, resulting in a demodulated output signal 253 (including a DC component that is proportional to the amplitude of the input signal 252), as similarly described earlier.

The perfusion detection device 200 is shown to also include a root mean square converter 242 configured to receive the demodulated signal 253 from the lock-in amplifier 244 and multiply it by the necessary factor (e.g., square root of two) to produce a output signal 254 corresponding to a peak value as opposed to the root mean square value. Any convenient root mean square converter may be employed. Of interest are commercially available root mean square converters, such as Linear Technology LTC 1966; and the like.

As shown in FIG. 2, perfusion detection device 200 also includes a low pass filter 240 configured to receive the demodulated signal 254 output by the root mean square converter 242. The low pass filter is configured to pass essentially the DC signal component 255 and to filter out AC signals at other frequencies. In certain embodiments, the DC signal is not a pure DC signal as noise at frequencies very close to the reference frequency may result in very low frequency AC outputs. Attenuation of these low frequency AC outputs, if present, depends upon the low pass filter bandwidth and rolloff. In certain instances, narrower bandwidths are employed so as to remove more of the noise having frequencies very close to the reference frequency. Any convenient low pass filter may be employed. Of interest are commercially available low pass filters, such as Analog Devices AD711 operational amplifier (with the appropriate resistors and capacitors to set filter wavelength), and the like.

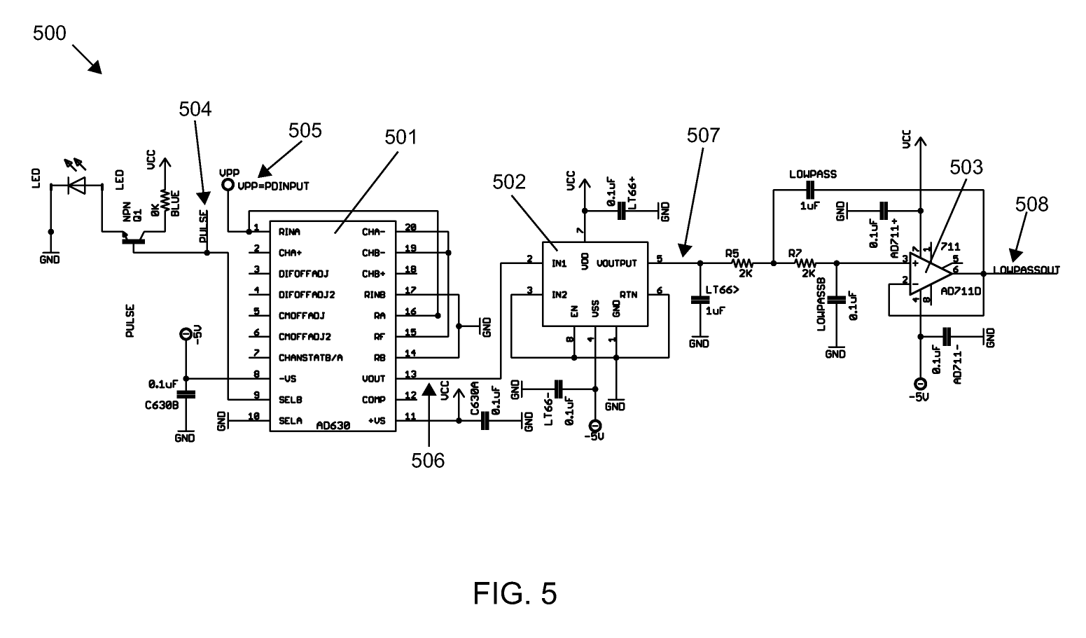

FIG. 5 illustrates a portion of a circuit schematic including a lock-in amplifier, root mean square converter, and low pass filter for a perfusion detection device, according to some embodiments. Circuit part 500 is shown to include lock-in amplifier 501, root mean square converter 502, and low pass filter 503. Lock-in amplifier is shown to receive a reference pulse (as represented by reference numeral 504) and input signal (as represented by reference numeral 505). Input signal 505 may correspond, for example, to output signal 450 shown in FIG. 4. Lock-in amplifier is coupled to root mean square converter 502 and is configured to generate a demodulated signal (as represented by reference numeral 506) that includes a DC signal component that is proportional to the input signal 505. Root mean square converter 502 converts the demodulated signal 506 from a root mean square value to a peak value, as represented by reference numeral 507. Low pass filter 503 is coupled to the root mean square converter 502 and filters signal 507 to block out AC signals and pass the DC signal component (as represented by reference numeral 508) that is proportional to the input signal 505. Additional circuitry also shown in FIG. 5 includes various circuitry components provided for proper operation of the lock-in amplifier 501, root mean square converter 502, and low pass filter 503—e.g., biasing circuitry, noise suppression circuitry, etc.

Turning back to FIG. 2, the perfusion detection device 200 is shown to further include an analog to digital converter 256 configured to receive the analog signal 255 from the low pass filter 240 and output a digital signal 257 to a functional block 258 that converts the digital signal 255 to the perfusion detection result 259. The perfusion detection result 259 may then be sent to a display 232 for display to the user.

Microcontroller 236 (e.g., ATMEGA328 from Atmel, San Jose, Calif.) may be configured to execute the function of functional block 258. In some embodiments, the processor is configured to provide a perfusion detection result in 1 minute or less following illumination of a target tissue site 250 with light from the LED 228 without pre-calibration. For example, the perfusion detection result 259 is obtained, in some instances, within 30 seconds or less, such as 10 seconds or less following illumination of a target tissue site 250 with light from the LED 228 without pre-calibration. In some embodiments, the perfusion detection result is obtained substantially immediately, such as 5 seconds or less, following illumination of the target tissue site 250 with the LED 228.

Microcontroller 236 may also execute various other functions of functional block 258 such as interpreting the strength of the fluorescent light, changing the interpretation of light emitted from the LED (e.g., LED brightness, modulation rate, etc.), monitoring battery level, indicating low battery level, power savings, etc. In some embodiments, the microcontroller 236 may be configured to simultaneously generate the pulse-wave and interpret the strength of the fluorescent light. Microcontroller 236 may, in some instances, be configured to provide software controlled analysis of fluorescence and software adjustment of fluorescence display.

Microcontroller 236 may also be configured to execute the functions of functional block 260 that performs adjustments to program settings of the device according to various user inputs. For example, the device may be programmed according to user inputs to adjust settings such as scale, screen brightness, etc.

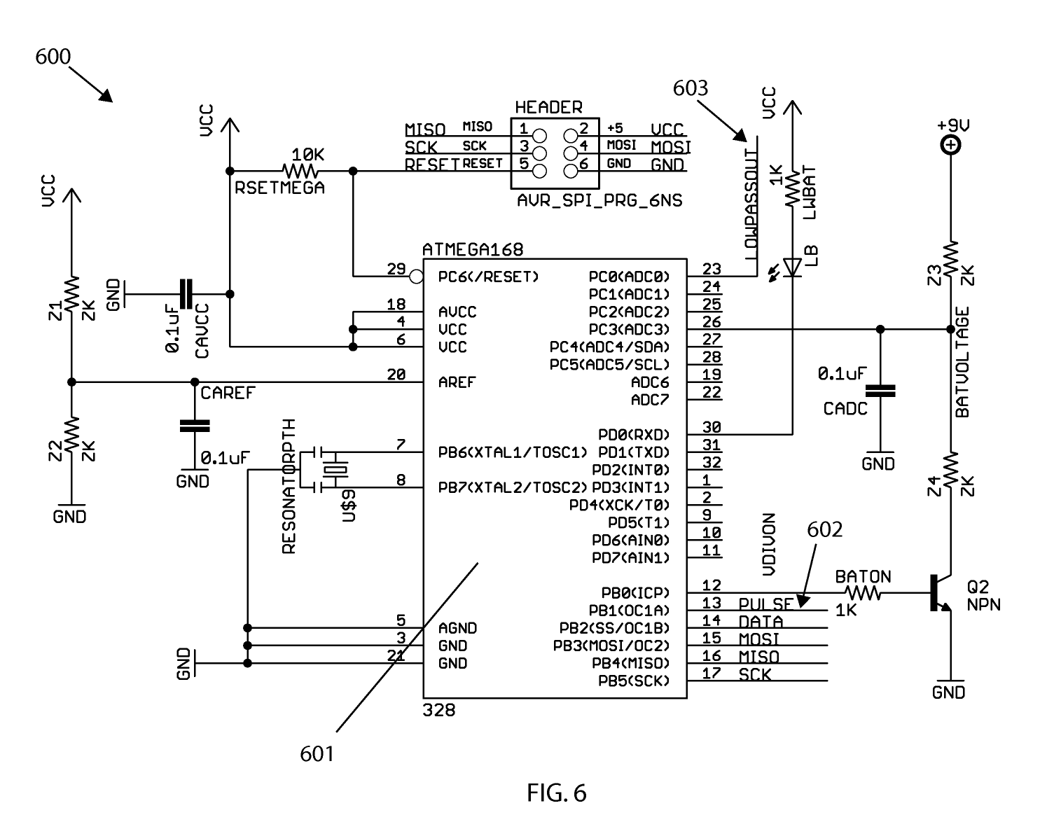

FIG. 6 illustrates a portion of a circuit schematic including a microcontroller for a perfusion detection device, according to some embodiments. Circuit part 600 is shown to include microcontroller 601. Microcontroller 601 is shown to generate a pulse-wave signal, as represented at reference numeral 602, that is supplied to an LED (not shown) to pulse the LED, and to lock-in amplifier as a reference signal (e.g., such as the reference signal represented at reference numeral 504 for lock-in amplifier 501 shown in FIG. 5). Further, microcontroller 603 is further shown to receive the signal output from a low pass filter (e.g., such as the signal output represented at reference numeral 508 for low pass filter 503 shown in FIG. 5). Microcontroller 603 is also shown to include signal lines 604 which may be coupled to a display (not shown), for example, to output the perfusion detection result to the display. Additional circuitry also shown in FIG. 6 includes various circuitry components provided for proper operation of the microcontroller 601—e.g., power circuitry, biasing circuitry, noise suppression circuitry, etc.

It should be understood that some of the techniques introduced above can be implemented by programmable circuitry programmed or configured by software and/or firmware, or they can be implemented entirely by special-purpose “hardwired” circuitry, or in a combination of such forms. Such special-purpose circuitry (if any) can be in the form of, for example, one or more application-specific integrated circuits (ASICS), programmable logic devices (PLDs), field-programmable gate arrays (FPGAs), etc.

Software or firmware implementing the techniques introduced herein may be stored on a machine-readable storage medium and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, personal digital assistant (PDA), manufacturing took, any device with one or more processors, etc.). For example, a machine-accessible medium includes recordable/non-recordable media (e.g., read-only memory (ROM); random access memory (RAM); magnetic disk storage media; optical storage media; flash memory devices; etc.), etc.

The term “logic”, as used herein, can include, for example, special purpose hardwired circuitry, software and/or firmware in conjunction with programmable circuitry, or a combination thereof.

### METHODS

Aspects of the invention also include methods of detecting perfusion in target tissue of a subject. Perfusion may be detected in a variety of different target tissues, including but not limited to replanted parts (e.g., such as fingers, facial parts, scalps, hands, arms, etc.); microvascular transplants (e.g., flaps); and the like.

With respect to detecting perfusion in target tissue of a subject, embodiments of such methods include administering a fluorescent agent to the subject. Various fluorescent agents may be used, such as fluorescein and derivatives thereof (e.g., fluorescein isothiocyanate (FITC)), rhodamine and derivatives thereof, e.g., tetramethylrhodamine isothiocyanate (TRITL), R-Phycoerythrin (RPE), etc. Administration may be via any convenient route, including but not limited to, intravenous, oral, etc., where the fluorescent agent may be formulated into a vehicle suitable for the intended delivery route, as desired.

Following agent administration, methods of the invention may further include waiting for an amount of time sufficient before obtaining a perfusion reading with a device according to the invention. This amount of time may vary and in some instances is an amount of time sufficient for the fluorescent agent to enter the target tissue assuming the target tissue is adequately perfused. The amount of time may vary depending on different factors, including but not limited to the nature of the fluorescent agent, the administration route, etc. In some instances, the amount of time ranges from 5 seconds to 10 minutes or more, such as 5 seconds to 5 minutes or more.

Following agent administration and any option time period (e.g., as described above), the methods may also include obtaining a perfusion detection result for the target tissue of interest from a hand-held perfusion detection device of the invention, e.g., as described above.

The obtaining of the perfusion detection result may include the step of positioning the device in operational relationship to the target tissue location (e.g., as shown in FIG. 1B where the speculum is position in close apposition to the target location (a finger tip)), and illuminating the target tissue location with light from the LED of the device. For example, in some instances, the perfusion detection device may be positioned such that the target tissue site is adjacent to an aperture in the housing—e.g., the head section of the housing. In some instances, the perfusion detection result is obtained within one minute or less following illumination of the target tissue with the LED. In some instances, the perfusion detection result is obtained substantially immediately following illumination of the target tissue site with the LED. For example, in some instances the perfusion detection result is obtained within five seconds or less following illumination of the target tissue with the LED.

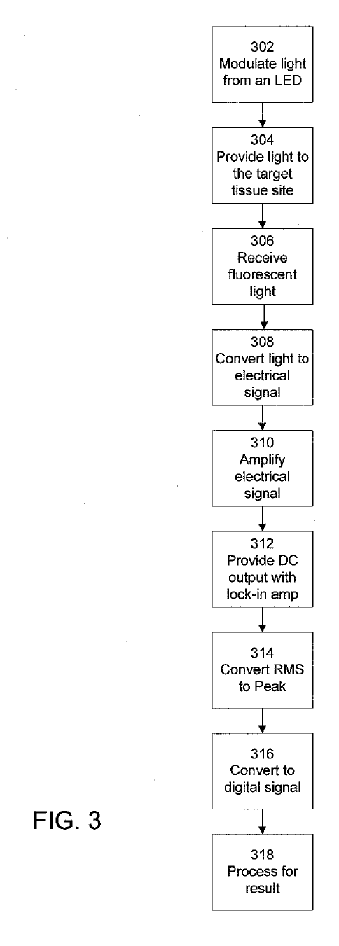

Aspects of the invention further include methods of obtaining a perfusion detection result, as represented in FIG. 3. FIG. 3 illustrates a method of detecting perfusion, according to some embodiment of the invention.

At block 302, light is modulated from an LED. For example, a pulse-wave may be generated by a pulse-wave generator and provided to the LED to pulse the LED on and off at a frequency pulse-wave. At block 304, the pulsating light from the LED is provided to the target tissue site. For example, the pulsating light is directed to the target tissue site by one or more fiber optics through an aperture. In some instances, the pulsating light is filtered using a narrowband filter tuned to the wavelength of the light emitted from the LED. For example, a blue light may be emitted from a blue LED and filtered by a narrowband filter tuned to pass the blue light. Upon illumination of the target tissue site with the light from the LED, any fluorescent agent diffused in the target tissue is excited and begins to fluoresce.

At block 306, the fluorescent light is received. For example, the fluorescent light may enter an aperture in the housing (e.g., in the head section of the housing) and travel through a receiving fiber optic to a detector (e.g., photodiode). In some instances, the method also includes filtering the fluorescent light. For example, a narrowband filter may be positioned between the receiving fiber optic and the photodiode to pass the fluorescent light and filter out any light with wavelengths outside of the narrow band.

At block 308, the fluorescent light is converted into an electrical energy signal representative of the fluorescent light by the detector. For example, the photodiode may receive the fluorescent light and generate an output current signal representative of the fluorescent light (i.e., pulsing at the same frequency). At block 310, the electrical energy signal is amplified.

At block 312, a DC output signal proportional to the amplitude of the output signal from the detector is provided using a lock-in amplifier. At block 314, the DC output signal is converted from a root mean square value to a peak value and passed through a low pass filter. The low pass filter passes the DC output signal and essentially filters out other AC signals that may be accompanying the DC output signal. At block 316, the DC output signal is converted into a digital signal using an ND converter. For example, a microprocessor may convert the received DC output signal from the root mean square converter into a digital signal representative of the DC output signal.

At block 318, the digital signal is processed by a functional block to determine a perfusion detection result. The functional block receives the digital signal representative of the DC output signal and processes the signal accordingly. The processing of the digital signal may include a processor executing an algorithm for determining a perfusion detection result. In block 320, the perfusion detection result is displayed on a display.

In some instances, the methods further include a surgical procedure, such as a microsurgical procedure, prior to detection of perfusion at the target tissue location. The specific surgical procedure may vary, where surgical procedures of interest include, but are not limited to: replantation of parts (e.g., fingers, hands, arms, facial parts, scalps, etc.) and microvascular transplant procedures).

Depending on the perfusion detection result that is obtained, methods of the invention may further include one or more perfusion remediation protocols, e.g., where the perfusion detection result indicates sub-optimal or no perfusion in the target tissue location. Perfusion remediation protocols that may be performed may vary, and include but are not limited to: removal of mechanical obstruction to flow (e.g., removal of tight dressings); pharmacologic therapy (e.g., in the form of administration of agents such as anticoagulants and medicinal leeching agents); surgical intervention (e.g., to re-establish vascular flow and remove occlusions); etc.

The subject methods are suitable for use with a variety of vascularized animals, such as mammal. Mammals of interest include, but are not limited to: race animals, e.g. horses, dogs, etc., work animals, e.g. horses, oxen etc., and primates, e.g., humans. In some embodiments, the mammals on which the subject methods are practiced are humans.

### UTILITY

The subject perfusion detections devices and methods find use in a variety of different applications where it is desirable to detect perfusion, including but not limited to surgical procedures (e.g., microsurgical procedures). Monitoring of blood flow, both arterial inflow and venous outflow, is especially critical in microsurgery. Microsurgery, also referred to as microvascular surgery, is surgery that is performed on very small structures, such as blood vessels and nerves, using specialized equipment and an operating microscope. For example, detection of proper blood flow is vital in replanted parts such as fingers, facial parts, scalps, hands, arms, legs, etc. Furthermore, detection of proper blood flow is vital in microvascular transplants, also known as “free flaps” or “flaps”. These types of surgeries generally include one or two small inflow arteries and one or two small outflow veins. Any compromise in circulation of the arterial inflow or venous outflow can lead to loss of the replanted part or flap. Early detection of arterial or venous compromise can be critical to salvaging replants and flaps, and to prevent failure. Accordingly, the devices and methods find use in the above, as well as other, procedures (e.g., in detecting levels of a fluorescent agent in other tissues and organs, such as muscle).

### KITS

Also provided are kits for use in practicing the subject methods, where the kits may include one or more of the above devices, and/or components of the subject systems, e.g., specula, dosages of fluorescent agent, LEDs (e.g., two or more LEDs of differing light emission properties), optical filters (e.g., two or more differing optical filters configured to be operably positioned in the device relative to the detector), etc., as described above. Various components may be packaged as desired, e.g., together or separately.

In addition to above mentioned components, the subject kits may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A hand-held perfusion detection device, said device comprising:
a power source;
a light source;
a detector; and
a processor configured to provide a perfusion detection result in 1 minute or less following illumination of a target tissue site with light from the light emitting diode without pre-calibration;
wherein the power source, light emitting diode, detector and processor are present in a self-contained pocket-sized housing.

2. The device according to claim 1, wherein the processor is configured to provide a perfusion detection result substantially immediately.

3. The device according to claim 2, wherein the light source is a light emitting diode.

4. The device according to claim 1, wherein the detector is a photodiode.

5. The device according to claim 4, wherein the device comprises a signal amplifier coupled to the photodiode.

6. The device according to claim 5, wherein the signal amplifier and photodiode are present in an EMI shield.

7. The device according to claim 1, wherein the device comprises a wave generator configured to provide a pulse-wave signal to the light emitting diode.

8. The device according to claim 7, wherein the device comprises a lock-in amplifier configured to receive a signal from the detector.

9. The device according to claim 8, wherein the lock-in amplifier is configured to receive a pulse-wave reference signal from the wave generator.

10. The device according to claim 8, wherein the device comprises a root mean square converter configured to receive a signal from the lock-in amplifier.

11. The device according to claim 10, wherein the device comprises low pass filter configured to receive a signal from the root mean square converter.

12. The device according to claim 11, wherein the processor comprises an analog to digital signal converter configured to receive a signal from the low pass filter and output a digital signal to a functional block that converts the digital signal to the perfusion detection result.

13. The device according to claim 12, wherein the processor further comprises the wave generator.

14. The device according to claim 13, wherein the device comprises a display configured to display the perfusion detection result.

15. The device according to claim 1, wherein the device comprises an interchangeable filter operably positioned relative to the detector.

16. The device according to claim 1, wherein the light emitting diode is interchangeable.

17. The device according to claim 1, wherein the processor comprises a wave generator having a frequency that is programmably adjustable.

18. The device according to claim 17, wherein the frequency can be programmably adjusted to frequency ranging from 1 Hz to 1 MHz.

19. The device according to claim 1, wherein the power source is a battery.

20. The device according to claim 1, wherein the housing comprises a body section and head section.

21. The device according to claim 20, wherein the head section is movable relative to the body section.

22. The device according to claim 21, wherein the head section comprises the detector and the body section comprises the power source, light emitting diode and processor.

23. The device according to claim 22, wherein the device is configured to emit and receive light through a common aperture.

24. The device according to claim 23, wherein the device comprises an ambient light shield configured to shield the detector from ambient light.

25. The device according to claim 24, wherein the ambient light shield is a speculum.

26. A method comprising:
administering a fluorescent agent to the subject; and
obtaining a perfusion detection result for target tissue from a hand-held perfusion detection device comprising:
a power source;
a light emitting diode;
a detector; and
a processor configured to provide a perfusion detection result in 1 minute or less following illumination of a target tissue site with light from the light emitting diode without pre-calibration;
wherein the power source, light emitting diode, detector and processor are present in a self-contained pocket-sized housing.

27. The method according to claim 26, wherein the fluorescent agent is administered to the subject by injection.

28. The method according to claim 26, wherein the fluorescent agent is fluorescein.

29. The method according to claim 16, wherein the perfusion detection result is obtained in 1 minute or less following illumination of the target tissue with the light emitting diode.

30. The method according to claim 29, wherein the perfusion detection result is obtained in 5 seconds or less following illumination of the target tissue with the light emitting diode.

31. A kit comprising:
(a) a hand-held perfusion detection device, said device comprising:
a power source;
a light emitting diode;
a detector; and
a processor configured to provide a perfusion detection result in 1 minute or less following illumination of a target tissue site with light from the light emitting diode without pre-calibration;
wherein the power source, light emitting diode, detector and processor are present in a self-contained pocket-sized housing; and
(b) instructions for using the device in a perfusion detection application.

32. The kit according to claim 31, wherein the kit further comprises a dosage of a fluorescent agent.

33. The kit according to claim 31, wherein the kit further comprises two or more light emitting diodes of differing light emission properties.

34. The kit according to claim 33, wherein the kit further comprises two or more differing optical filters configured to be operably positioned in the device relative to the detector.
